(19) United States
(12) Patent Application Publication
(10) Pub. No.: US 2024/0415993 A1
(43) Pub. Date: Dec. 19, 2024

(54) UVC Sterilization Chamber

(71) Applicant: UVSee Inc., Winnipeg (CA)

(72) Inventors: Zoran Bilic, Winnipeg (CA); Milos Lekic, Winnipeg (CA)

(21) Appl. No.: 18/700,813

(22) PCT Filed: Oct. 12, 2022
(86) PCT No.: PCT/CA2022/051496

§ 371 Date: Apr. 12, 2024

Related U.S. Application Data
(60) Provisional application No. 63/254,698, filed on Oct. 12, 2021.

(51) Int. Cl. A61L 2/10; A61L 2/24; A61L 2/26

(52) U.S. Cl. CPC A61L 2/10; A61L 2/24; A61L 2/26; A61L 2202/11; A61L 2202/121; A61L 2202/122; A61L 2202/14; A61L 2202/24

(57) ABSTRACT

Described herein is a microbial sterilization device comprising an inner chamber or reactor that utilizes the Ultra Violet C spectrum (UVC) for the sterilization of small instruments and articles, as discussed herein. The UVC sterilization device 1 comprises an inner reactor 10 defined by reflective inner surfaces, a UVC transparent platform and at least one UVC lamp.

### PRIOR APPLICATION INFORMATION

The instant application claims the benefit of U.S. Provisional Patent Application Ser. No. 63/254,698, filed Oct. 12, 2021 and entitled “UVC STERILIZATION CHAMBER”, the entire contents of which are incorporated herein by reference for all purposes.

### BACKGROUND OF THE INVENTION

Currently, steam sterilization, in the form of a small autoclave, is used by many businesses, such as doctors, dentists, offices, tattoo parlours and hair salons, for the sterilization of small articles.

While widely used, autoclaving is not without its drawbacks. Sterilization times for certain types of instruments can be as long as 30 minutes and even once the sterilization is complete, the instrument needs cooling time prior to use. Thereby leading to long wait periods for completion of the sterilization process and subsequent cooling time. Furthermore, some materials are not suited for repeated exposure to the combination of pressure and moisture, meaning that repeated autoclave sterilization may in fact hasten degradation or loss of integrity of some articles requiring frequent sterilization.

Clearly, an effective, more convenient method for sterilization of instruments and small articles is needed. As such, an Ultra violet spectrum C (UVC) device comprised of distinct design features was developed so as to provide an alternative, highly efficient method of sterilization.

### SUMMARY OF THE INVENTION

In one embodiment of the invention, there is provided an UVC sterilization device comprising of:
an inner reactor formed by a plurality of UVC reflective surfaces, said inner reactor comprising at least one UVC lamp and an UVC transparent platform for placement of items to be sterilized thereon; and
an outer shell comprising of a door for accessing the inner reactor and control panel.

According to another aspect of the invention, there is provided a method for sterilizing an article comprising:
providing a UVC sterilization device comprising:
   an inner reactor formed by a plurality of UVC reflective surfaces, said inner reactor comprising at least one UVC lamp and a UVC transparent platform for placing items to be sterilized thereon; and
   an outer shell comprising a door for accessing the inner reactor;
placing the article to be sterilized on the UVC transparent platform; and activating the UVC sterilization device.

### BRIEF DESCRIPTION OF THE DRAWINGS

FIG. 1 is a side view of the UVC sterilization device.

FIG. 2 is an exploded view of the reactor or core of the UVC sterilization device.

### DESCRIPTION OF THE PREFERRED EMBODIMENTS

Unless defined otherwise, all technical and scientific terms used herein have the same meaning as commonly used vernacular. Although any methods and materials similar or equivalent to those described herein can be used in the practice or testing of the present invention, the preferred methods and materials are now presented.

Described below is a microbial sterilization device comprising an inner chamber or reactor that utilizes the Ultra Violet C spectrum (UVC) for the sterilization of small instruments and articles, as discussed herein.

According to an aspect of the invention, there is provided a UVC sterilization device comprising:
an inner reactor formed by a plurality of UVC reflective surfaces, said inner reactor comprising at least one UVC lamp and a UVC transparent platform for placing items to be sterilized thereon; and
an outer shell comprising a door for accessing the inner reactor.

In some embodiments, the UVC sterilization device further comprises a microprocessor unit configured for measuring UV light intensity during sterilization and monitoring internal air temperature within the inner reactor.

In some embodiments of the invention, the at least one UVC lamp is configured to turn off when the door is opened.

In some embodiments of the invention, the at least one UVC lamp comprises a first UVC lamp at a top of the inner reactor and a second UVC lamp at a bottom of the inner reactor. The UVC transparent platform may be positioned between the first UVC lamp and the second UVC lamp.

The at least one UVC lamp may be a mercury tube or an excimer tube.

The UVC transparent platform may be composed of a UVC transparent glass, for example, a fused silica or a synthetic silica quartz.

The UVC transparent platform may be approximately 5 mm thick, for example, between 4.5-5.5 mm thick.

In some embodiments of the invention, the plurality of UVC reflective surfaces are arranged such that the inner reactor has a cylindrical, substantially cylindrical, or polygonal shape.

In some embodiments of the invention, the plurality of UVC reflective surfaces are selected from the group consisting of: aluminum or steel sheet covered with PTFE; aluminum or steel sheet coated with barium sulphate; polished aluminum; and polished steel.

According to another aspect of the invention, there is provided a method for sterilizing an article comprising:
providing a UVC sterilization device comprising:
   an inner reactor formed by a plurality of UVC reflective surfaces, said inner reactor comprising at least one UVC lamp and a UVC transparent platform for placing items to be sterilized thereon; and
   an outer shell comprising a door for accessing the inner reactor;
placing the article to be sterilized on the UVC transparent platform; and
activating the UVC sterilization device.

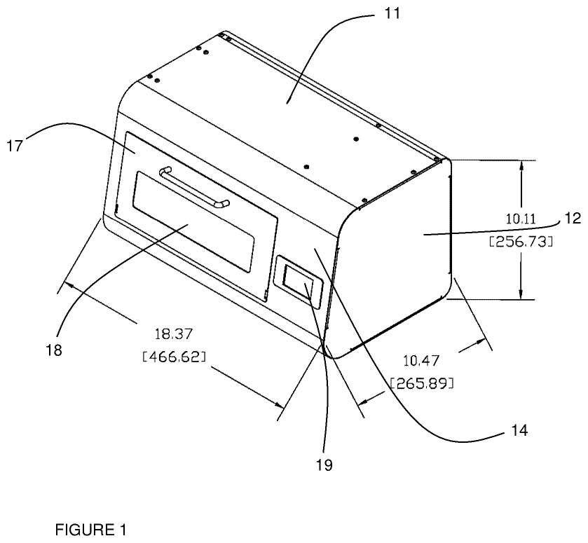

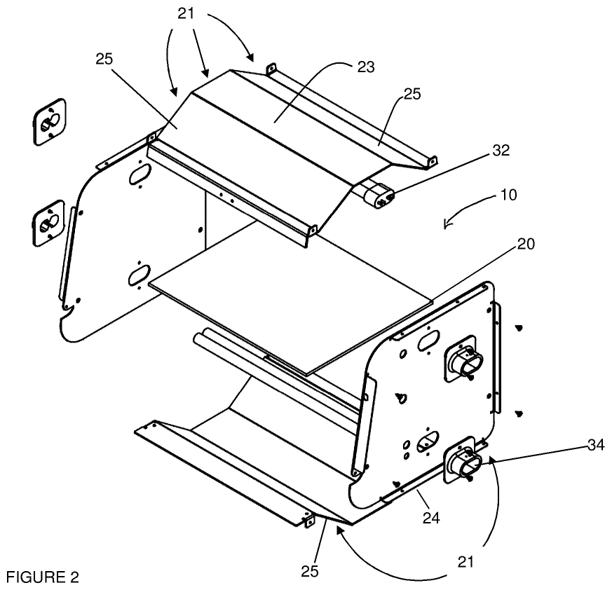

With reference to the drawings, more specifically to the aforementioned FIGS. 1-2, the UVC sterilization device 1 comprises an inner reactor 10 defined by reflective surfaces 21, a UVC transparent platform 20, at least one UVC lamp 32,34 and a door 17 for accessing the inner reactor.

In the embodiment shown in FIG. 1, the inner reactor 10 is defined broadly by top panel 11, side panels 12, front face 14, a rear panel and a bottom panel.

In the embodiment shown in FIG. 1, the front face 14 includes a door 17 having a window 18 and a control panel 19.

In the embodiment shown in FIG. 2, the inner reactor 20 is defined by or formed by the reflective surfaces 21 and further includes an upper UVC lamp 32 proximal to the top plate 23 and a lower UVC lamp 34 proximal to the bottom plate 24 and the platform 20 is positioned between the two lamps 32,34 as discussed herein.

As will be appreciated, items to be sterilized are placed on the platform 20. Ideally, the items to be sterilized would be suspended in midair for the sterilization process. Since that is not possible, in some embodiments, the platform is composed of UVC transparent glass, such as—by no means limited to—fused silica or synthetic silica quartz. Other suitable UVC transparent glass can be utilized and would be within the scope of the invention.

In other embodiments of the invention, the platform 20 comprises a grille made of steel or aluminum that is structured with minimal contact surface so as to avoid contact points on the platform. Contact points on the platform 20 of this type would prevent full exposure to the UVC light leading to interference with the sterilization process, unless the respective contact surfaces of the platform 20 are minimized. A platform that has sufficiently little respective contact surfaces minimizing any shading or prevention of UVC light exposure can be considered to be UVC transparent.

The thickness of the stage (platform) would be deemed suitable for use when it allows sufficient UVC light to pass through the stage and sterilize the underside of the objects on the platform, that is, the side of the object(s) resting or positioned on the stage. In some embodiments of the invention, the stage or platform is approximately or about 5 mm thick, that is, between 4.5-5.5 mm thick.

The combination of the UVC transparent platform and the presence of UVC lamps at the top and bottom of the inner reactor promote full exposure of items placed on the platform to the UVC light. Specifically, most of the UVC light flux from the UVC lamps is focused at the UVC transparent platform as a result of this arrangement.

In some embodiments of the invention, the surfaces that define or form the inner reactor are composed of a suitable material, such as, a steel or aluminum.

The surface of inner reactor in some embodiments of the invention are covered with a highly reflective UVC material such as PTFE, aka Teflon®. Alternatively, barium sulphate, which is a mineral powder that reflects UVC, could be considered for the reflective surface. In other embodiments, the UVC reflective surfaces may be polished aluminum or polished steel. Other suitable surfaces for reflecting UVC light will be apparent to those of skill in the art and are within the scope of the invention.

Teflon provides >95% reflection if it is high density, compressed. Regular virgin Teflon reflects >90%, while if the Teflon is etched on one side, probably >93%. In comparison, polished aluminum reflects >70%.

In some embodiments of the invention, the UVC reflective surfaces are coated with Teflon, as Teflon provides diffused reflection which means there will be no shadow formation inside of the chamber, which can significantly reduce radiation exposure.

Furthermore, as a result of positioning the platform between the two UVC light sources and the use of reflective surfaces in forming the inner reactor, most of the UVC light flux is focused to the platform where items for sterilization will be placed.

The design of the invention is such that the UVC reflective surfaces within the inner reactor form substantially a cylindrical shape giving the best reflection. While not wishing to be bound to a particular theory or hypothesis, the inventors believe that due to the elongated shape of the UVC lamps, a cylinder would be the best shape, followed by a polygonal cylinder and lastly, a square/rectangular profile. Ultimately, a square or rectangular profile for the inner reactor will work, provided the UVC light source is sufficiently strong, as will be discussed herein.

In the embodiment shown in FIG. 2, the inner reactor is defined by or formed by a top plate 23, bottom plate 24, and four side plates 25. As discussed herein, this shape can be considered to be substantially cylindrical in that a shape such as this will provide suitable reflection.

While a true cylindrical shape is optimal for maximum UV light flux, manufacturing a “true” cylindrical shape is not cost effective and can be difficult for production. “True” cylindrical shapes are within the definition of “substantially cylindrical shapes” as defined above and therefore within the scope of the invention. In the examples provided herein, and for manufacturing purposes, an octagonal shape was chosen.

The wavelength range of UVC light is generally accepted as 100-280 nm. As such, in some embodiments of the invention, the UVC lamp is any suitable light source that produces ultraviolet light within the wavelength range of 100-280 nm.

There are 2 maxima in UVC spectrum that affect microorganism DNA, 220 nm and 265 nm. Specifically, UVC light at these wavelengths is believed to promote the formation of crosslinks between adjacent nucleotides within a nucleic acid molecule.

In some embodiments of the invention, at least one of the UVC lamps is a mercury tube, that either produce 254 nm and 180 nm simultaneously or one that is arranged such that the tube glass is reflective for 180 nm, thereby preventing ozone formation.

In other embodiments, at least one of the UV lamps is an excimer tube that produces monochromatic light at 222 nm. More specifically, such a tube or lamp produces UVC light at a wavelength that is close to 220 nm and most importantly is apparently is not harmful to human skin and eyes.

In some embodiments of the invention, the source of UVC light is for example a 254 nm mercury germicidal lamp. The lamps can individually be either 60 watts or 95 watts electrical power which roughly produces 20 or 30 watts of 254 nm light emission. Efficiency of these lamps is about 35%, meaning about ⅓ of the electrical power been converted into 254 nm light.

As can be seen in FIG. 2, in some embodiments, the UVC lamps extend along the length of the inner reactor.

As can be seen in FIG. 2, the inner reactor is separated into two halves by the stage or platform for placing items to be sterilized thereon, as discussed herein.

As discussed below, the objects for sterilization are placed on the upper surface of the stage or platform. Because of the substantially cylindrical space and the use of the reflective coating on the surfaces of the side walls, the top and the bottom of the reactor that define the substantially cylindrical space, the objects are simultaneously sterilized from all sides and angles, which eliminates the need for rotation and repositioning of the object to achieve the desired sterilization level. Essentially, the sterilization process for a given object is continuous and need not be interrupted for repositioning and prevents recontamination.

Furthermore, the light flux density inside of the reactor is high due to its shape and its highly reflective surfaces (more than 95% reflection). Consequently, the process does not require longer than 120 seconds of sterilization time in order to achieve >99.9999% sterilization efficacy.

In use, the UVC sterilization device 1 is opened via the door 17 and the objects to be sterilized are placed on the UVC transparent platform 20. The light exposure time is controlled by a microprocessor unit connected to the control pad 19. As a result of this arrangement, the exposure time of the items to be sterilized to the UVC lamps is precisely controlled. Additionally, the microprocessor unit measures the UV light intensity during sterilization and monitors the internal air temperature. The purpose of this measurement is to ensure that the lamps always operate at the optimal efficiency and gives the user a warning if the light intensity increases or decreases

Essentially, it is the energy (mJ/cm²) that kills the microbes, so mW/cm²*sec=mJ/cm². It is to be noted that exposure of a UV dose of 10-15 mJ/cm² is sufficient to produce a log reduction of most bacteria of log₁₀=5. Radiation output measurement provides very important feedback information. Specifically, this report ensures that radiation energy density, mJ/cm², is always above the minimum requirement to guarantee sterilization. If lamp output drops below a minimum level, the microprocessor will notify the user that sterilization is unreliable or not possible.

As discussed above, access to the inner reactor 10 is through the front door 17, which has a fail-safe mechanism that prevents the user from accidental exposure to harmful light. Essentially, the lamps turn off automatically when the user opens the door and interrupts the process.

In some embodiments, the germicidal lamps are energized by an electromagnetic ballast that can be powered from 50/60 Hz, 85-270 Volts AC. Similarly, the control unit which houses the microprocessor can be powered from the same AC source. The reactor, control units, ballast, and all wiring are housed inside a steel enclosure. The entire unit is earth grounded to the AC cable when plugged into a power outlet.

In some embodiments of the invention, as noted in FIG. 1, the reactor access door 17 is made of steel and has a borosilicate glass window 18 for visual observation of the sterilization. This glass completely blocks harmful UV light, but allows passage of some blue light. As well, the door completely prevents leakage of any harmful UV light.

It is to be appreciated that any suitably sized items can be sterilized in the UVC sterilization device. These items would include: cellphones, glasses, keys, remote controls, small hand tools like hair salon tools, scissors, tweezers, combs, all household cutlery, tattoo parlour needles and tools, and office items like pens, pencils, customer gifts with smooth surfaces, business cards, credit cards, paper money and coins. Basically, anything with a smooth surface that might exchange hands can be placed in the device. That is, the UVC sterilization device can be used to sterilize any item currently sterilized by a small autoclave unit, but with the distinct advantages that the sterilization time is much shorter (2 minutes or less) and that the items being sterilized are not subjected to steam and pressure. As such, items that are water-labile and/or heat labile can be sterilized with the UVC sterilization device. Furthermore, the items can be used immediately following cessation of the sterilization process in the UVC device as the items do not need to cool.

While the preferred embodiments of the invention have been described above, it will be recognized and understood that various modifications may be made therein, and the appended claims are intended to cover all such modifications which may fall within the spirit and scope of the invention.

1. A UVC sterilization device comprising:
an inner reactor having an octagonal shape, said octagonal shape formed by a plurality of UVC reflective surfaces selected from the group consisting of: aluminum or steel sheet covered with PTFE; aluminum or steel sheet coated with barium sulphate; polished aluminum; and polished steel, said inner reactor comprising a first UVC lamp at a top of the inner reactor and a second UVC lamp at a bottom of the inner reactor and a platform for placing items to be sterilized thereon positioned between the first UVC lamp and the second UVC lamp, said platform comprising a UVC transparent platform or a grille made of steel or aluminum; and
an outer shell comprising a door for accessing the inner reactor.

2. The UVC sterilization device according to claim 1 further comprising a microprocessor unit configured for measuring UV light intensity during sterilization and monitoring internal air temperature within the inner reactor.

3. The UVC sterilization device according to claim 1 wherein the first UVC lamp and the second UVC lamps are configured to turn off when the door is opened.

4. (canceled)

5. (canceled)

6. The UVC sterilization device according to claim 1 wherein at least one UVC lamp is a mercury tube or an excimer tube.

7. The UVC sterilization device according to claim 1 wherein the UVC transparent platform comprises a UVC transparent glass.

8. The UVC sterilization device according to claim 7 wherein the UVC transparent glass is fused silica or synthetic silica quartz.

9. (canceled)

10. The UVC sterilization device according to claim 1 wherein the platform is between 4.5-5.5 mm thick.

11. (canceled)

12. (canceled)

13. A method for sterilizing an article comprising:
providing a UVC sterilization device comprising:
an inner reactor having an octagonal shape formed by a plurality of UVC reflective surfaces selected from the group consisting of: aluminum or steel sheet covered with PTFE; aluminum or steel sheet coated with barium sulphate; polished aluminum; and polished steel, said inner reactor comprising a first UVC lamp at a top of the inner reactor and a second UVC lamp at a bottom of the inner reactor and a platform positioned between the first UVC lamp and the second UVC lamp for placing items to be sterilized thereon, said platform comprising a UVC transparent platform or a grille made of steel or aluminum; and
an outer shell comprising a door for accessing the inner reactor;
placing the article to be sterilized on the platform; and
activating the UVC sterilization device.

14. The method according to claim 8 further comprising a microprocessor unit configured for measuring UV light intensity during sterilization and monitoring internal air temperature within the inner reactor.

15. The method according to claim 13 wherein the first UVC and the second UVC lamps are configured to turn off when the door is opened.

16. (canceled)

17. (canceled)

18. The method according to claim 13 wherein at least one UVC lamp is a mercury tube or an excimer tube.

19. The method according to claim 13 wherein the UVC transparent platform comprises a UVC transparent glass.

20. The method according to claim 19 wherein the UVC transparent glass is fused silica or synthetic silica quartz.

21. (canceled)

22. The method according to claim 13 wherein the platform is between 4.5-5.5 mm thick.

23. (canceled)

24. (canceled)
